(19) United States
(12) Patent Application Publication
(10) Pub. No.: US 2024/0110930 A1
(43) Pub. Date: Apr. 4, 2024

(54) METHODS, ASSAYS AND COMPOSITIONS FOR MEASURING BRAIN DAMAGE OR HARM DURING SURGERY

(71) Applicants: NanoSomiX, Inc., Aliso Viejo, CA (US); National Cerebral and Cardiovascular Center, Osaka (JP)

(72) Inventors: Masato Mitsuhashi, Irvine, CA (US); Takahiro Tadokoro, San Diego, CA (US); Kenji Yshitani, Kashiba (JP)

(21) Appl. No.: 18/275,602

(22) PCT Filed: Feb. 3, 2022
(86) PCT No.: PCT/US2022/015155

§ 371 Date: Aug. 2, 2023

Related U.S. Application Data
(60) Provisional application No. 63/144,985, filed on Feb. 3, 2021.

(51) Int. Cl. G01N 33/68; A61K 31/4152; A61K 45/06

(52) U.S. Cl. CPC G01N 33/6896; A61K 31/4152; A61K 45/06; G01N 2333/70596; G01N 2800/52

(57) ABSTRACT

The present disclosure relates to methods and assays for quantifying brain-derived exosomes in biological samples from subjects before, during, and after surgery. The disclosure also provides methods and compositions for measuring and/or prognosing brain damage during surgery. The compositions and methods of the disclosure are also useful for preventing and/or treating brain damage in subjects undergoing surgery and predicting postoperative cognitive dysfunction (POCD) and/or predicting postoperative delirium (POD).

### RELATED APPLICATIONS

This application claims priority to U.S. Provisional Patent Application Ser. No. 63/144,985, filed on Feb. 3, 2021, which is hereby incorporated by reference herein in its entirety.

### FIELD OF THE INVENTION

The present disclosure relates to methods and assays for quantifying brain-derived exosomes in biological samples from subjects before, during, and after surgery. The disclosure also provides methods and compositions for measuring and/or prognosing brain damage during surgery. The compositions and methods of the disclosure are also useful for preventing and/or treating brain damage in subjects undergoing surgery and predicting postoperative cognitive dysfunction (POCD) and/or predicting postoperative delirium (POD).

### BACKGROUND OF THE INVENTION

Postoperative cognitive dysfunction or decline (POCD) is defined as a prolonged cognitive function impairment that occurs within weeks to months following a surgical procedure. The rates of major complications (e.g., mortality) following cardiac surgery are currently low because of improvements in medical and surgical techniques, however, the incidence of POCD is still unchanged, and has become the most common postoperative complication. The reported incidence of POCD varies widely depending on the definition, test methods, and time of postoperative assessment. The incidence of POCD after cardiac surgery is reported to be 30-80% a few weeks after surgery and 10-60% after 3-6 months. The occurrence of POCD is associated with markedly adverse outcomes, such as prolonged hospitalization and rehabilitation, increased mortality, diminished quality of life, and more common working disability and early retirement, thereby producing a significant burden on the healthcare system.

Brain-derived exosomes/vesicles (e.g., neuron-derived, astrocyte-derived, and oligodendrocyte-derived exosomes/vesicles) referred to herein as BDE are present in biological samples and increases in their levels may be used to assess damage to brain tissue during or following surgery. Normal levels of BDE in biological samples following surgery have not been established. Thus, there is a need in the art for methods for quantifying BDE in biological samples and assessing changes in BDE levels as a result of surgery.

Postoperative delirium (POD) is a post-operative condition in which subjects have altered consciousness, orientation, memory, perception, and behavior. POD is not rare and is one of the common problems experienced with various types of surgeries. POD is associated with poor outcomes including functional and cognitive decline, longer hospitalization, institutionalization, greater costs, and higher mortality. Moreover, POD also increases the workload of the nursing staff. Thus, the prediction and prevention of POD is extremely important for anesthesiologists, surgeons, and hospitalists to maintain the quality of patient care by reducing overall healthcare cost. If it is predicted before an operation, surgeons may consider less invasive procedures, anesthesiologists may modify anesthesiology protocols, and nursing staff may prepare for POD management. Moreover, neurologists may become actively involved in the patient's care with various prophylactic treatments to prevent much more problematic post-operative cognitive decline (POCD). Therefore, the prediction of POD is a substantial unmet need in anesthesiology

Further, there is a need in the art for methods for quantifying BDE and compositions for measuring brain damage during surgery and post operatively. Additionally, there is a need in the art for compositions for quantifying exosomes as well as compositions and methods useful for treating neurological disease associated with cardiac surgery and other cardiac procedures. The disclosure meets this need by providing accurate, noninvasive methods for measuring changes in BDE levels and predicting and/or prognosing brain damage associated with surgery. The methods of the disclosure are useful for predicting, diagnosing, or prognosing postoperative cognitive decline (POCD) and/or post-operative delirium (POD) in subject undergoing or having undergone cardiac surgery. The disclosure further provides novel methods, assays, kits, and compositions for quantifying exosomes in biological samples from subjects that have undergone cardiac surgery. The disclosure also provides compositions and methods that are also useful for preventing and/or treating brain damage in subjects undergoing surgery.

### SUMMARY OF THE INVENTION

The present disclosure relates to novel methods, compositions, and kits for detecting and quantitating exosomes in a sample from a subject that has undergone surgery. In some embodiments, the disclosure provides a method comprising: (i) obtaining a biological sample from a subject that is undergoing or has undergone surgery, and (ii) detecting brain-derived exosomes in the sample by contacting the sample with antibodies and detecting binding between the brain-derived exosomes and the antibodies. In other embodiments, the detecting binding between the brain-derived exosomes and the antibodies further comprises contacting the brain-derived exosomes with a second antibody. In other embodiments, the antibodies are anti-CD171 antibodies, anti-EAAT1 antibodies, and anti-MOG antibodies. In still other embodiments, the second antibody is an anti-CD9 antibody, an anti-CD63 antibody, or an anti-CD81 antibody. In some embodiments, the method further comprises quantifying the levels of brain-derived exosomes in the biological sample. In other embodiments, the exosomes are selected from the group consisting of neuron-derived exosomes, astrocyte-derived exosomes, oligodendrocyte-derived exosomes, and microglia-derived exosomes. In yet other embodiments, the subject has been diagnosed or is suspected of having brain damage. In still other embodiments, the brain damage is postoperative cognitive dysfunction or decline (POCD). In some embodiments, the brain damage is postoperative delirium (POD). In other embodiments, the biological sample is selected from the group consisting of whole blood, serum, plasma, urine, interstitial fluid, peritoneal fluid, cervical swab, tears, saliva, buccal swab, skin, brain tissue, and cerebrospinal fluid. In some embodiments, the surgery is cardiac surgery. In other embodiments, the surgery is open heart surgery. In still other embodiments, the surgery is transcatheter aortic valve replacement (TAVR) or total aortic valve replacement (TAR). In yet other embodiments, the surgery further comprises transcatheter, endoscopic, or laparoscopic procedure.

The disclosure provides novel methods, compositions, and kits for detecting and quantitating brain derived exosomes (BDE) in a sample from a subject that is undergoing or has undergone surgery. In some embodiments, the disclosure provides a method comprising: (i) obtaining a biological sample from a subject that is undergoing or has undergone surgery, and (ii) detecting BDE in the sample by contacting the sample with antibodies that bind BDE and detecting binding between the BDE and the antibodies. In other embodiments, the detecting binding between the brain-derived exosomes and the antibodies further comprises contacting the BDE with a second antibody. In other embodiments, the antibodies are anti-CD171, anti EAAT1, anti-MOG and/or neuron selective BDE antibodies. In some embodiments, the exosomes are vesicles. In still other embodiments, the second antibody is an anti-CD9 antibody, an anti-CD63 antibody, and/or an anti-CD81 antibody. In some embodiments, the method further comprises quantifying the levels of BDE in the biological sample. In other embodiments, the exosomes are selected from the group consisting of neuron-derived exosomes, astrocyte-derived exosomes, and oligodendrocyte-derived exosomes. In yet other embodiments, the subject has been diagnosed or is suspected of having or developing brain damage postoperatively or postoperative cognitive dysfunction or decline (POCD). In yet other embodiments, the subject has been diagnosed or is suspected of having or developing postoperative delirium (POD). In other embodiments, the biological sample is selected from the group consisting of whole blood, serum, plasma, urine, interstitial fluid, peritoneal fluid, cervical swab, tears, saliva, buccal swab, skin, brain tissue, and cerebrospinal fluid. In some embodiments, the surgery is cardiac surgery. In some embodiments, the brain-derived exosomes include at least two exosomes from the group consisting of neuron-derived exosomes, astrocyte-derived exosomes, oligodendrocyte-derived exosomes, and microglia-derived exosomes. In other embodiments, the method further comprises determining the ratio of two exosomes from the group consisting of neuron-derived exosomes, astrocyte-derived exosomes, oligodendrocyte-derived exosomes, and microglia-derived exosomes. In some embodiments, the ratio of exosomes is increased or decreased compared to a control or predetermined value. In other embodiments, a lower ratio of NDE/ADE and/or ODE/ADE prior to surgery indicates that the subject will have postoperative delirium.

In other embodiments, the disclosure provides a method for measuring and/or detecting brain damage in a subject undergoing a surgical procedure comprising: (i) obtaining one or more biological samples from a subject before, during, and/or after undergoing surgery or any combination thereof, (ii) detecting BDE in the one or more samples by contacting the one or more samples with antibodies and detecting binding between the brain-derived exosomes and the antibodies, and (iii) comparing the levels of brain-derived exosomes in the one or more samples to a control sample and/or same subject sample obtained at a different time point in the surgical procedure. In some embodiments, the levels of BDE are increased or reduced compared to a control or subject sample obtained at a different point in the procedure. In other embodiments, the antibodies are anti-CD171, anti EAAT1, anti-MOG and/or other neuronal selective antibodies. In still other embodiments, the second antibody is an anti-CD9, an anti-CD63 antibody, and/or an anti-CD81 antibody and/or other exosome specific antibody. In some embodiments, the method further comprises quantifying the levels of BDE in the biological sample. In other embodiments, the exosomes are selected from the group consisting of neuron-derived exosomes, astrocyte-derived exosomes, oligodendrocyte-derived exosomes, and microglia-derived exosomes. In yet other embodiments, the subject has been diagnosed or is suspected of having brain damage. In still other embodiments, the brain damage is postoperative cognitive dysfunction or decline (POCD). In yet other embodiments, the subject has been diagnosed or is suspected of having or developing postoperative delirium (POD). In other embodiments, the biological sample is selected from the group consisting of whole blood, serum, plasma, urine, interstitial fluid, peritoneal fluid, cervical swab, tears, saliva, buccal swab, skin, brain tissue, and cerebrospinal fluid. In some embodiments, the surgery is cardiac surgery. In other embodiments, the method further comprises determining the ratio of two exosomes from the group consisting of neuron-derived exosomes, astrocyte-derived exosomes, oligodendrocyte-derived exosomes, and microglia-derived exosomes. In some embodiments, the ratio of exosomes is increased or decreased compared to a control or predetermined value. In other embodiments, a lower ratio of NDE/ADE and/or ODE/ADE prior to surgery indicates that the subject will have postoperative delirium.

In other embodiments, the disclosure provides a method for identifying subjects for preventing, minimizing, or treating damage to a brain during surgery, comprising: (i) obtaining a biological sample from a subject that is undergoing or has undergone surgery, (ii) detecting whether a brain-derived exosome is present in the sample by contacting the sample with an antibody and detecting binding between the brain-derived exosome and the antibody, and (iii) administering an effective amount of a compound to prevent, minimize, or treat damage to the brain. In some embodiments, the biological sample is selected from the group consisting of whole blood, serum, plasma, urine, interstitial fluid, peritoneal fluid, cervical swab, tears, saliva, buccal swab, skin, brain tissue, and cerebrospinal fluid. In other embodiments, the compound is selected from the group consisting of an anti-inflammatory compound, an anti-oxidative stress compound, a free radical scavenger compound, and a neuronal growth and survival factor compound. In some embodiments, the free radical scavenger compound is edaravone. In yet other embodiments, the compound is administered before, during, or after the surgery. In still other embodiments, the compound is administered if brain-derived exosomes are detected in the sample. In some embodiments, the compound is administered if brain-derived exosomes are increased or reduced in the sample compared to a control. In other embodiments, the exosomes are selected from the group consisting of neuron-derived exosomes, astrocyte-derived exosomes, oligodendrocyte-derived exosomes, and microglia-derived exosomes. In still other embodiments, the biological sample is selected from the group consisting of whole blood, serum, plasma, urine, interstitial fluid, peritoneal fluid, cervical swab, tears, saliva, buccal swab, skin, brain tissue, and cerebrospinal fluid. In other embodiments, the method further comprises determining the ratio of two exosomes from the group consisting of neuron-derived exosomes, astrocyte-derived exosomes, oligodendrocyte-derived exosomes, and microglia-derived exosomes. In some embodiments, the ratio of exosomes is increased or decreased compared to a control or predetermined value. In other embodiments, the compound is administered if the levels of brain-derived exosomes are increased or decreased compared to a control or predetermined value. In other embodiments, a lower ratio of NDE/ADE and/or ODE/ADE prior to surgery indicates that the subject will have postoperative delirium.

In other embodiments, the disclosure provides a method for predicting, diagnosing, and/or prognosing postoperative cognitive dysfunction or decline (POCD) or postoperative delirium (POD) in a subject, comprising: (i) obtaining a biological sample from a subject that is undergoing or has undergone surgery, (ii) detecting one more brain-derived exosomes in the sample, and (iii) comparing the levels of brain-derived exosomes in the sample to a control sample or value, thereby predicting, diagnosing, and/or prognosing postoperative cognitive dysfunction or decline (POCD) or postoperative delirium (POD) in the subject. In some embodiments, the biological sample is selected from the group consisting of whole blood, serum, plasma, urine, interstitial fluid, peritoneal fluid, cervical swab, tears, saliva, buccal swab, skin, brain tissue, and cerebrospinal fluid. In some embodiments, the exosomes are selected from the group consisting of neuron-derived exosomes, astrocyte-derived exosomes, oligodendrocyte-derived exosomes, and microglia-derived exosomes. In still other embodiments, the biological sample is selected from the group consisting of whole blood, serum, plasma, urine, interstitial fluid, peritoneal fluid, cervical swab, tears, saliva, buccal swab, skin, brain tissue, and cerebrospinal fluid. In some embodiments, the brain-derived exosomes include at least two exosomes from the group consisting of neuron-derived exosomes, astrocyte-derived exosomes, oligodendrocyte-derived exosomes, and microglia-derived exosomes. In other embodiments, the method further comprises determining the ratio of two exosomes from the group consisting of neuron-derived exosomes, astrocyte-derived exosomes, oligodendrocyte-derived exosomes, and microglia-derived exosomes. In some embodiments, the ratio of exosomes is increased or decreased compared to a control value or predetermined value. In some embodiments, the subject has undergone or is undergoing cardiac surgery. In other embodiments, the surgery is open heart surgery. In still other embodiments, the surgery is transcatheter aortic valve replacement (TAVR) or total aortic valve replacement (TAR). In yet other embodiments, the surgery further comprises transcatheter, endoscopic, or laparoscopic procedure.

In some embodiments, the disclosure provides a kit for measuring brain damage in a subject undergoing or having undergone surgery, the kit comprising one or more agents which specifically bind brain-derived exosomes, one or more containers for collecting and or holding the biological sample, and an instruction for its use. In some embodiments, the agent is an anti-CD9 antibody, an anti-CD171 antibody, an anti EAAT1 antibody, an anti-MOG antibody, an anti-CD63 antibody, and/or an anti-CD81 antibody. In other embodiments, the exosomes are selected from the group consisting of neuron-derived exosomes, astrocyte-derived exosomes, oligodendrocyte-derived exosomes, and microglia-derived exosomes. In still other embodiments, the surgery is cardiac surgery.

In some embodiments, the disclosure provides methods for detecting brain damage from surgery in a subject with at least 40%, 50%, 60%, 70%, 75%, 80%, 85%, 90%, 95%, 99%, or 100% specificity. In some embodiments, the disclosure provides methods for detecting brain damage from surgery in a subject with at least 40%, 50%, 60%, 70%, 75%, 80%, 85%, 90%, 95%, 99%, or 100% sensitivity. In other embodiments, the disclosure provides methods for detecting brain damage from surgery in a subject with at least 40%, 50%, 60%, 70%, 75%, 80%, 85%, 90%, 95%, 99%, or 100% accuracy.

These and other embodiments of the present disclosure will readily occur to those of skill in the art in light of the disclosure herein, and all such embodiments are specifically contemplated.

Each of the limitations of the disclosure can encompass various embodiments of the disclosure. It is, therefore, anticipated that each of the limitations of the disclosure involving any one element or combinations of elements can be included in each aspect of the disclosure. This disclosure is not limited in its application to the details of construction and the arrangement of components set forth in the following description. The disclosure is capable of other embodiments and of being practiced or of being carried out in various ways. Also, the phraseology and terminology used herein is for the purpose of description and should not be regarded as limiting. The use of “including,” “comprising,” or “having,” “containing”, “involving”, and variations thereof herein, is meant to encompass the items listed thereafter and equivalents thereof as well as additional items. It must be noted that as used herein and in the appended claims, the singular forms “a,” “an,” and “the” include plural references unless context clearly dictates otherwise.

### BRIEF DESCRIPTION OF THE DRAWINGS

FIG. 1 sets forth data showing an embodiment of an assay of the disclosure.

FIGS. 2A-2B set forth data showing assay reproducibility in relative light units (RLU) for an exemplary ELISA of the present disclosure. Panel 2A shows anti-CD171 NDE capture replicate results with multiple plasma samples vs 2B showing similar reproducible lack of activity using an IgG control antibody.

FIG. 3 sets forth data showing plasma levels of neuron derived exosomes (NDE) in preoperative subjects. Y axis shows U/mL using the plasma dilution curve of a standard plasma control which is arbitrarily assigned 100 U/mL.

FIGS. 4A-4B set forth data showing post-operative changes in neuron-derived exosomes (NDE) plasma units per ml levels in transcatheter aortic valve implantation (TAVI) subjects (FIG. 4A) and total arterial revascularization (TAR) subjects (FIG. 4B).

FIGS. 5A-5I set forth data showing changes in plasma levels of NDE (neuron derived exosomes), ADE (astrocyte derived exosomes), and ODE (oligodendrocyte derived exosomes) after TAR procedure. Blood samples were obtained before and after operation as well as at 1, 2, and 5 days after operation. U/mL and log U/ml are shown for NDE (AB), ADE (DE), and ODE (GH). Panel A,D, and G show subjects who did not develop POD and panels B,E and H show subjects who developed POD during the post-operative 5 days. Panels C,F, and G: show data when Log(U/mL) of post-operative samples was subtracted from log(U/mL) of the pre-operative samples (Δ log). Δ represents the mean minus standard deviation of POD(−) (n=11) and represents the mean plus standard deviation of POD(+) subjects (n=7).

FIGS. 6A-6C set forth data showing plasma levels of NDE, ADE, and ODE in subjects that developed POD (+) and subjects that did not develop POD (−). Y axis shows U/mL using the plasma dilution curve of a standard plasma control which is arbitrarily assigned 100 U/mL.

FIGS. 7A-7E set forth data showing the ratio of NDE/ADE (A), ODE/NDE (B), and ODE/ADE (C). POD prediction among TAR subjects was analyzed by ROC (D-E) with an AUC score of 81% and 84% for NDE/ADE and ODE/ADE, respectively.

FIGS. 8A-8G set forth data showing assay validation. A: Capture antibody specificity. Mouse monoclonal antibody (IgG2a) against human CD171, control mouse IgG, and control mouse IgG2a were immobilized on the ELISA wells. Three different control human plasma (light, medium, and dark gray columns) and diluents alone (white columns, invisible) were applied to the designated wells and ELISA was carried out using anti-human CD9 probes. B: Probe specificity. Three control plasma (light, medium, and dark gray columns) and buffer alone (white column, invisible) were applied to mouse IgG and anti-CD171-immobilized ELISA wells, respectively. Then, each well was reacted with biotinylated probes of mouse IgG, mouse IgG2b, and anti-human CD9 (mouse monoclonal IgG2b). C. Exosome specificity I: exosome reduction. Plasma samples were incubated with anti-CD81−(dark gray columns) and control mouse IgG-immobilized paramagnetic beads (light gray columns), respectively, then supernatant plasma samples were applied to 3 different ELISA wells, where anti-CD171, anti-CD81−, and control mouse IgG were previously immobilized. After each well was washed extensively, each well was reacted with biotinylated anti-CD9 to complete sandwich ELISA. D. Exosome specificity II: Spiked exosomes. Exosomes were prepared from control plasma by ExoQuick, and suspended in PBS. Three different diluted exosomes and PBS were added into plasma or plasma diluents, and [CD171+CD9+] was determined. E. Plasma dilution studies. Three control plasma samples were diluted in PBS (closed and open symbols) and applied to mouse IgG (dotted lines) and anti-CD171-immobilized ELISA wells (solid lines). Then, ELISA was run. F. Intra-assay reproducibility. The assay was always run in duplicate, and the variation of 2 replicates (RLU) were shown (n=134 subjects). Each dot represents a single individual. G. Inter-assay reproducibility. Three different control plasma samples were analyzed 12 times separately. Using the first data as 100%, % Change in each experiment was shown.

FIGS. 9A-9E set forth data showing exosome validation. A-D. SEM. Plasma samples were applied to anti-CD171 (A-B) and anti-CD81-immobilized ELISA wells (C-D) with exactly same protocol as our ELISA. After each well was washed extensively with PBS, each well bottom was punched out, suspended in fix solution, and analyzed by SEM. E. NTA. Plasma samples were applied to anti-CD171- and control mouse IgG-immobilized paramagnetic beads. Captured exosomes were eluted by low pH 2.5 solution, neutralized, then particle size distribution and concentrations were analyzed by NanoSight NS300. Solid line is the eluent from anti-CD171 immobilized paramagnetic beads, and dotted line is the eluent from control IgG-bound paramagnetic beads.

FIG. 10 sets forth data showing range of control values. Total 192 subjects from teens' to >80 years olds were used to determine [CD171+CD9+]. Y-axis was U/ml in log scale. Bars are median values in each group, and statistical analysis (Student's t-test) was carried out using log values

FIGS. 11A-11C set forth data showing monitoring plasma levels of [CD171+CD9+]. Y axis is % change in U/ml from the first blood sample in each subject and X axis was the period (hours to years) of blood sample collection. A: high school cross-country athletes (non-contact sports, n=9). Blood was drawn once a month from July to November. B: high school American football athletes (contact sports, n=22). Blood was drawn once a month from July to November. C: adult soccer players (n=19). Blood samples were collected before and 2, 24, and 48 hours after intensive heading practice

### DESCRIPTION OF THE INVENTION

It is to be understood that the disclosure is not limited to the particular methodologies, protocols, cell lines, assays, and reagents described herein, as these may vary. It is also to be understood that the terminology used herein is intended to describe particular embodiments of the present disclosure, and is in no way intended to limit the scope of the present disclosure as set forth in the appended claims.

It must be noted that as used herein and in the appended claims, the singular forms “a,” “an,” and “the” include plural references unless context clearly dictates otherwise. Thus, for example, a reference to “a fragment” includes a plurality of such fragments; a reference to an “antibody” is a reference to one or more antibodies and to equivalents thereof known to those skilled in the art, and so forth.

Unless defined otherwise, all technical and scientific terms used herein have the same meanings as commonly understood by one of ordinary skill in the art to which this disclosure belongs. Although any methods and materials similar or equivalent to those described herein can be used in the practice or testing of the present disclosure, the preferred methods, devices, and materials are now described. All publications cited herein are incorporated herein by reference in their entirety for the purpose of describing and disclosing the methodologies, reagents, and tools reported in the publications that might be used in connection with the disclosure. Nothing herein is to be construed as an admission that the disclosure is not entitled to antedate such disclosure by virtue of prior disclosure.

The practice of the present disclosure will employ, unless otherwise indicated, conventional methods of chemistry, biochemistry, molecular biology, cell biology, genetics, immunology and pharmacology, within the skill of the art. Such techniques are explained fully in the literature. See, e.g., Gennaro, A. R., ed. (1990) Remington's Pharmaceutical Sciences, 18th ed., Mack Publishing Co.; Colowick, S. et al., eds., Methods In Enzymology, Academic Press, Inc.; Handbook of Experimental Immunology, Vols. I-IV (D. M. Weir and C. C. Blackwell, eds., 1986, Blackwell Scientific Publications); Maniatis, T. et al., eds. (1989) Molecular Cloning: A Laboratory Manual, 2nd edition, Vols. I-III, Cold Spring Harbor Laboratory Press; Ausubel, F. M. et al., eds. (1999) Short Protocols in Molecular Biology, 4th edition, John Wiley & Sons; Ream et al., eds. (1998) Molecular Biology Techniques: An Intensive Laboratory Course, Academic Press); PCR (Introduction to Biotechniques Series), 2nd ed. (Newton & Graham eds., 1997, Springer Verlag).

The disclosure relates, in part, to the discovery that brain-derived exosomes can be assayed to identify subjects that have suffered or may suffer brain damage or harm and/or postoperative cognitive dysfunction or decline (POCD) or postoperative delirium (POD) from surgery.

The disclosure is based, in part, on the discovery of unexpected abnormal levels in certain brain-derived exosomes present in the circulation of subjects having undergone surgery (e.g., cardiac surgery). The disclosure demonstrates that brain-derived exosome levels may be assayed to predict, diagnose, and/or prognose brain damage or harm and/or postoperative cognitive dysfunction or decline (POCD) or postoperative delirium (POD) in a subject that has undergone surgery. The disclosure further shows that measurement of brain-derived exosomes from a subject may be used to predict the subsequent development of a postoperative cognitive dysfunction or decline (POCD) (e.g., identify a subject at risk of developing POCD) or postoperative delirium (POD) (e.g., identify a subject at risk of developing POD).

The present disclosure also provides compositions for use in the methods described herein. Such compositions may include small molecule compounds; peptides and proteins including antibodies or functionally active fragments thereof; and polynucleotides including small interfering ribonucleic acids (siRNAs), micro-RNAs (miRNAs), ribozymes, and anti-sense sequences. (See, e.g., Zeng (2003) Proc Natl Acad Sci USA 100:9779-9784; and Kurreck (2003) Eur J Biochem 270:1628-1644.)

The present disclosure further provides kits for measuring brain damage or harm in a subject undergoing or having undergone surgery. In these embodiments, the kits comprise one or more antibodies which specifically bind brain-derived exosomes, one or more containers for collecting and or holding the biological sample, and an instruction for the kits use.

The section headings are used herein for organizational purposes only, and are not to be construed as in any way limiting the subject matter described herein.

### Biological Sample

The present disclosure provides methods and assays for quantifying brain-derived exosomes in biological samples from subjects before, during, and after surgery. The present disclosure also provides methods and compositions for measuring brain damage or harm during surgery. Brain-derived exosome levels are determined in a biological sample obtained from a subject before, during, and/or after surgery or any combination thereof. In some embodiments, the biological sample of the disclosure is blood. In some embodiments, about 1-10 mL of blood is drawn from a subject. In other embodiments, about 10-50 mL of blood is drawn from a subject. Blood can be drawn from any suitable area of the body, including an arm, a leg, or blood accessible through a central venous catheter. In some embodiments, blood is collected before, during, or after surgery. The timing of collection can also be coordinated to increase the number and/or composition of exosomes present in the sample. For example, blood can be collected following exercise or a treatment that induces vascular dilation.

Blood may be combined with various components following collection to preserve or prepare samples for subsequent techniques. For example, in some embodiments, blood is treated with an anticoagulant, a cell fixative, a protease inhibitor, a phosphatase inhibitor, a protein, a DNA, or an RNA preservative following collection. In some embodiments, blood is collected via venipuncture using vacuum collection tubes containing an anticoagulant such as EDTA or heparin. Blood can also be collected using a heparin-coated syringe and hypodermic needle. Blood can also be combined with components that will be useful for cell culture. For example, in some embodiments, blood is combined with cell culture media or supplemented cell culture media (e.g., cytokines).

Biological samples can also be obtained from other sources known in the art, including whole blood, serum, plasma, urine, interstitial fluid, peritoneal fluid, cervical swab, tears, saliva, buccal swab, skin, cerebrospinal fluid, or other tissues including, for example, brain tissues.

### Enrichment or Isolation of Exosomes

Samples can be enriched for exosomes through positive selection, negative selection, or a combination of positive and negative selection. In some embodiments, exosomes are directly captured. In other embodiments, blood cells are captured and exosomes are collected from the remaining biological samples. In some embodiments, the brain-derived exosomes enriched in the biological samples are neuron-derived exosomes, astrocyte-derived exosomes, oligodendrocyte-derived exosomes, and/or microglia-derived exosomes.

Samples can also be enriched for brain-derived exosomes based on differences in the biochemical properties of exosomes. For example, samples can be enriched for exosomes based on antigen, nucleic acid, metabolic, gene expression, or epigenetic differences. In some of the embodiments based on antigen differences, antibody-conjugated magnetic or paramagnetic beads in magnetic field gradients or fluorescently labeled antibodies with flow cytometry are used. In some of the embodiments based on nucleic acid differences, flow cytometry is used. In some of the embodiments based on metabolic differences, dye uptake/exclusion measured by flow cytometry or another sorting technology is used. In some of the embodiments based on gene expression, cell culture with cytokines is used. Samples can also be enriched for exosomes based on other biochemical properties known in the art. For example, samples can be enriched for exosomes based on pH or motility. Further, in some embodiments, more than one method is used to enrich for exosomes. In other embodiments, samples are enriched for exosomes using antibodies, ligands, or soluble receptors.

In other embodiments, surface markers are used to positively enrich exosomes in the sample. In other embodiments, NCAM, CD9, CD63, CD81, neuron-specific enolase, diverse neuron or astrocyte adhesive proteins, microglial CD18/11, or CD3 T cell membrane cell surface markers are used to enrich for exosomes. In other embodiments, SNAP25, CD171, EAAT1, OMGP, DR1, SR2A, SR2C, GABAB1, GluR-1, KOR, OR, or DAT are used to enrich for exosomes. In some embodiments, cell surface markers that are not found on exosomes populations are used to negatively enrich exosomes by depleting cell populations. Flow cytometry sorting may also be used to further enrich for exosomes using cell surface markers or intracellular or extracellular markers conjugated to fluorescent labels. Intracellular and extracellular markers may include nuclear stains or antibodies against intracellular or extracellular proteins preferentially expressed in exosomes. Cell surface markers may include antibodies against cell surface antigens that are preferentially expressed on exosomes (e.g., NCAM). In some embodiments, the cell surface marker is a neuron-derived exosome surface marker, including, for example, NCAM or CD171, CD9, SNAP25, EAAT1, OMGP, DR1, SR2A, SR2C, GABAB1, GluR-1, KOR, OR, or DAT. In some embodiments, a monoclonal NCAM, CD9, CD63, CD81, neuron-specific enolase, CD171, SNAP25, EAAT1, OMGP, DR1, SR2A, SR2C, GABAB1, GluR-1, KOR, OR, or DAT antibody is used to enrich or isolate exosomes from the sample. In certain aspects, the NCAM, CD9, CD63, CD81, neuron-specific enolase, CD171, SNAP25, EAAT1, OMGP, DR1, SR2A, SR2C, GABAB1, GluR-1, KOR, OR, or DAT antibody is biotinylated. In this embodiment, biotinylated NCAM or CD171 antibody can form an antibody-exosome complex that can be subsequently isolated using streptavidin-agarose resin or beads. In other embodiments, the NCAM, CD9, CD63, CD81, neuron-specific enolase, CD171, SNAP25, EAAT1, OMGP, DR1, SR2A, SR2C, GABAB1, GluR-1, KOR, OR, or DAT antibody is a monoclonal anti-human NCAM, CD9, CD63, CD81, neuron-specific enolase, CD171, SNAP25, EAAT1, OMGP, DR1, SR2A, SR2C, GABAB1, GluR-1, KOR, OR, or DAT antibody.

In some embodiments, enriched exosomes from the biological sample are subsequently enriched for a specific type of brain-derived exosome (e.g., a subpopulation of brain-derived exosomes). For example, the biological sample is enriched for exosomes and then the enriched exosomes are subsequently enriched for neural-derived exosomes. In some embodiments, the biological sample is enriched for individual neural cell sources of exosomes. In certain aspects, the neural cell sources of exosomes are microglia, neurons, or astrocytes. In other embodiments, surface markers are used to enrich for a specific type of exosome (e.g., neural-derived exosome). In some embodiments, CD171, CD9, SNAP25, EAAT1, OMGP, DR1, SR2A, SR2C, GABAB1, GluR-1, KOR, OR, and/or DAT cell surface markers are used to enrich for a specific type of exosome. In some embodiments, cell surface markers that are not found on the exosomes of interest are used to negatively enrich exosomes by depleting unwanted cell populations. Flow cytometry sorting may also be used to further enrich for specific types of exosomes using cell surface markers or intracellular or extracellular markers conjugated to fluorescent labels. Intracellular and extracellular markers may include nuclear stains or antibodies against intracellular or extracellular proteins preferentially expressed in or on the exosomes of interest. Cell surface markers may include antibodies against cell surface antigens that are preferentially expressed on exosomes (e.g., CD171, CD9, SNAP25, EAAT1, OMGP, DR1, SR2A, SR2C, GABAB1, GluR-1, KOR, OR, and DAT). In some embodiments, the cell surface marker is a neuron-derived exosome surface marker, including, for example, CD171. In some embodiments, the cell surface marker is an astrocyte-derived exosome surface marker, including, for example, EAAT1. In some embodiments, the cell surface marker is an oligodendrocyte-derived exosome surface marker, including, for example, OMG. In some embodiments the cell surface marker is a receptor for dopamine, serotonin, GABA, glutamate, opioid, orexin, adrenalin, noradrenalin, acetylcholine, and/or dopamine transporter. In some embodiments, a monoclonal CD171, CD9, SNAP25, EAAT1, OMG, DR1, SR2A, SR2C, GABAB1, KOR, OR, or DAT antibody is used to enrich or isolate exosomes from the sample. In certain aspects, the CD171, CD9, SNAP25, EAAT1, OMGP, DR1, SR2A. SR2C, GABAB1, GluR-1, KOR, OR, or DAT antibody is biotinylated. In this embodiment, biotinylated CD171, CD9, SNAP25, EAAT1, OMGP, DR1, SR2A, SR2C, GABAB1, GluR-I, KOR, OR, or DAT antibody can form an antibody-exosome complex that can be subsequently isolated using streptavidin-agarose resin or beads. In other embodiments, the CD171, CD9, SNAP25, EAAT1, OMGP, DR1, SR2A, SR2C, GABAB1, GluR-1, KOR, OR, or DAT antibody is a monoclonal anti-human CD171, CD9, SNAP25, EAAT1, OMGP, DR1, SR2A, SR2C, GABA 1, GluR-1, KOR, OR, or DAT antibody.

In other embodiments, brain-derived exosomes are isolated or enriched from a biological sample comprising: contacting a biological sample with an agent under conditions wherein an exosome present in said biological sample binds to said agent to form an exosome-agent complex; and isolating said exosome from said exosome-agent complex to obtain a sample containing said exosome, wherein the purity of exosomes present in said sample is greater than the purity of exosomes present in said biological sample. In certain embodiments, the agent is an antibody or a lectin. Lectins useful for forming an exosome-lectin complex are described in U.S. Patent Application Publication No. 2012/0077263. In some embodiments, the brain-derived exosomes are neuron-derived exosomes, astrocyte-derived exosomes, oligodendrocyte-derived exosomes, or microglia-derived exosomes. In some embodiments, multiple isolating or enriching steps are performed. In certain aspects of the present embodiment, a first isolating step is performed to isolate exosomes from a blood sample and a second isolating step is performed to isolate neural-derived exosomes from other exosomes. In other embodiments, the exosome portion of the exosome-agent complex is lysed using a lysis reagent and the protein levels of the lysed exosome are assayed. In some embodiments, the antibody-exosome complex is created on a solid phase. In yet other embodiments, the methods further comprise releasing the exosome from the antibody-exosome complex. In certain embodiments, the solid phase is non-magnetic beads, magnetic beads, agarose, or sepharose. In other embodiments, the exosome is released by exposing the antibody-exosome complex to low pH between 3.5 and 1.5. In yet other embodiments, the released exosome is neutralized by adding a high pH solution. In other embodiments, the released exosome is lysed by incubating the released exosomes with a lysis solution. In still other embodiments, the lysis solution contains inhibitors for proteases and phosphatases.

### Brain Damage or Harm

The disclosure provides methods for measuring, diagnosing, and/or prognosing brain damage or harm in a subject undergoing or having undergone surgery. Brain damage is an injury that causes the destruction or deterioration or harmful alteration of brain cells. Brain injuries can occur during surgery if the patient's brain is deprived of oxygen for a long enough period of time or as a result of toxicity from anesthesia. When this occurs, it may be a result of severe surgical trauma, the anesthetic itself, administering incorrect dosage of anesthesia due to a misreading of the medical record entry error, miscalculation of the patient's size, weight and mass, selection of the wrong anesthetic agent, length of time under anesthesia, use and duration of cardiopulmonary by-pass or other invasive procedures

Delirium is an acute state of confusion that results in inattention and cognitive failure. Medical or surgical conditions can often trigger delirium. Delirium in surgical subjects is linked to nervous tissue injury in the brain that can lead to loss of cognitive ability (e.g., postoperative delirium, POD).

Cognitive dysfunction is the most common clinical evidence of brain injury after cardiac surgery. It can be detected with neuropsychological testing by a trained examiner Disturbances in memory, psychomotor speed, executive function, visuo-constructional ability, and ability to concentrate are characteristics of brain injury after surgery. Post-operative cognitive dysfunction (POCD) may be detected in 14% to 48% of subjects before hospital discharge after cardiac surgery. Moreover, impairment continues to be detectable in at least 30% for 6 weeks and in 25% for 6 months. (Selnes et al. Lancet 1999; 353: 1601; and van Dijk et al. J Thorac Cardiovasc Surg. 2000; 120: 632.)

In some embodiments, the present disclosure enables a medical practitioner to measure, diagnose, and/or prognose brain damage in a subject during and/or after surgery. In other embodiments, the present disclosure enables a medical practitioner to rule out or eliminate brain damage as a diagnostic possibility. In yet other embodiments, the present disclosure enables a medical practitioner to identify a subject at risk of developing brain damage after surgery. In other embodiments, the present disclosure enables a medical practitioner to predict whether a subject will later develop postoperative cognitive dysfunction or decline (POCD) or postoperative delirium (POD). In further embodiments, the present disclosure enables a medical practitioner to prescribe a therapeutic regimen or predict benefit from therapy in a subject undergoing or having undergone surgery. In some embodiments, the surgery is cardiac surgery (e.g., total arterial revascularization or transcatheter aortic valve replacement).

In some embodiments, an increase in brain-derived exosomes in the circulation during or following surgery indicates brain damage is occurring or has occurred. The proportional change in brain-derived exosomes in circulation is associated with brain damage severity including delirium and postoperative cognitive dysfunction or decline.

### Brain-Derived Exosomes and Antigens

Brain-derived exosome levels are assayed in a biological sample obtained from a subject having or at-risk of having brain damage during surgery. Brain-derived exosomal antigens may be used to detect or measure brain-derived exosome levels in biological samples. In some embodiments, the antigen is a receptor for dopamine, serotonin, GABA, glutamate, opioid, orexin, adrenalin, noradrenalin, acetylcholine, and/or dopamine transporter. In some embodiments, the antigen is CD9, CD61, CD81, CD171, SNAP25, EAAT1, OMG, DR1, SR2A, SR2C, GABAB1, GluR-1, KOR, OR, and/or DAT. Other known antigens may be used in combination with the antigens of the present disclosure. Examples of such antigens are provided in US Patent Application Pub. No. 2015/0119278, the contents of which are hereby incorporated by reference.

One of ordinary skill in the art has several methods and devices available for the detection and analysis of the brain-derived of the instant disclosure. With regard to exosome levels in patient test samples, immunoassay devices and methods are often used. These devices and methods can utilize labeled molecules in various sandwich, competitive, or non-competitive assay formats, to generate a signal that is related to the presence or amount of an analyte of interest. Additionally, certain methods and devices, such as biosensors and optical immunoassays, may be employed to determine the presence or amount of analytes without the need for a labeled molecule.

Preferably the brain-derived exosomes are analyzed using an immunoassay, although other methods are well known to those skilled in the art. The presence or amount of a brain-derived exosome is generally determined using antibodies specific for each marker and detecting specific binding. Any suitable immunoassay may be utilized, for example, enzyme-linked immunoassays (ELISA), radioimmunoassay (RIAs), competitive binding assays, planar waveguide technology, and the like. Specific immunological binding of the antibody to the marker can be detected directly or indirectly. Direct labels include fluorescent or luminescent tags, metals, dyes, radionuclides, and the like, attached to the antibody. Indirect labels include various enzymes well known in the art, such as alkaline phosphatase, horseradish peroxidase and the like.

The use of immobilized antibodies specific for the brain-derived exosomes is also contemplated by the disclosure. The antibodies could be immobilized onto a variety of solid supports, such as magnetic or chromatographic matrix particles, the surface of an assay place (such as microtiter wells), pieces of a solid substrate material (such as plastic, nylon, paper), and the like. An assay strip could be prepared by coating the antibody or a plurality of antibodies in an array on solid support. This strip could then be dipped into the test sample and then processed quickly through washes and detection steps to generate a measurable signal, such as a colored spot.

The analysis of a plurality of brain-derived exosomes may be carried out separately or simultaneously with one test sample. Several markers may be combined into one test for efficient processing of a multiple of samples. In addition, one skilled in the art would recognize the value of testing multiple samples (for example, at successive time points) from the same individual. Such testing of serial samples will allow the identification of changes in brain-derived exosomes levels over time. Increases or decreases in brain-derived exosomes levels, as well as the absence of change in marker levels, would provide useful information about the disease status that includes, but is not limited to identifying the approximate time from onset of the event, the presence and amount of salvageable tissue, the appropriateness of drug therapies, the effectiveness of various therapies, identification of the severity of the event, identification of the disease severity, and identification of the patient's outcome, including risk of future events.

An assay consisting of a combination of the brain-derived exosomes referenced in the instant disclosure may be constructed to provide relevant information related to differential diagnosis. Such a panel may be constructed using 1, 2, 3, 4, or 5, or more or individual brain-derived exosomes. The analysis of a single brain-derived exosomes or subsets of brain-derived exosomes comprising a larger panel of brain-derived exosomes could be carried out with methods described within the instant disclosure to optimize clinical sensitivity or specificity in various clinical settings. In some embodiments, the brain-derived exosomes are selected from the group consisting of neuron-derived exosomes, astrocyte-derived exosomes, oligodendrocyte-derived exosomes, and microglia-derived exosomes. In some embodiments, the ratio of two exosomes from the group consisting of neuron-derived exosomes, astrocyte-derived exosomes, oligodendrocyte-derived exosomes, and microglia-derived exosomes are determined.

The analysis of brain-derived exosome levels could be carried out in a variety of physical formats as well. For example, the use of microtiter plates or automation could be used to facilitate the processing of large numbers of test samples. Alternatively, single sample formats could be developed to facilitate immediate treatment and diagnosis in a timely fashion, for example, in ambulatory transport or emergency room settings. Particularly useful physical formats comprise surfaces having a plurality of discrete, addressable locations for the detection of a plurality of different analytes. Such formats include protein microarrays, or “protein chips” and capillary devices.

Brain-derived exosomes of the present disclosure serve an important role in the early detection and monitoring of brain damage during and/or following surgery. Brain-derived exosomes associated with brain damage are typically found in a bodily sample that can be measured. The measured amount can correlate to underlying disorder or disease pathophysiology, presence or absence of brain damage, probability of postoperative cognitive dysfunction or decline (POCD) or postoperative delirium (POD) in the future. In subjects receiving treatment for their condition the measured amount will also correlate with responsiveness to therapy. In some embodiments, an increase in the level of one or more brain-derived exosomes of the present disclosure is indicative of brain damage. Accordingly, the methods of the present disclosure are useful for the diagnosis of brain damage.

In some embodiments, the levels of brain-derived exosomes are measured by a method selected from the group consisting of immunohistochemistry, immunocytochemistry, immunofluorescence, immunoprecipitation, western blotting, and ELISA.

### Clinical Assay Performance

The methods of the present disclosure may be used in clinical assays to measure, diagnose, and/or prognose brain damage in a subject undergoing or having undergone surgery. Clinical assay performance can be assessed by determining the assay's sensitivity, specificity, area under the ROC curve (AUC), accuracy, positive predictive value (PPV), and negative predictive value (NPV). Disclosed herein are assays for measuring, diagnosing, and/or prognosing brain damage in a subject undergoing or having undergone surgery.

The clinical performance of the assay may be based on sensitivity. The sensitivity of an assay of the present disclosure may be at least about 40%, 45%, 50%, 55%, 60%, 65%, 70%, 75%, 80%, 85%, 90%, 95%, 99%, or 100%. The clinical performance of the assay may be based on specificity. The specificity of an assay of the present disclosure may be at least about 40%, 45%, 50%, 55%, 60%, 65%, 70%, 75%, 80%, 85%, 90%, 95%, 99%, or 100%. The clinical performance of the assay may be based on area under the ROC curve (AUC). The AUC of an assay of the present disclosure may be at least about 0.5, 0.55, 0.6, 0.65, 0.7, 0.75, 0.8, 0.85, 0.9, or 0.95. The clinical performance of the assay may be based on accuracy. The accuracy of an assay of the present disclosure may be at least about 40%, 45%, 50%, 55%, 60%, 65%, 70%, 75%, 80%, 85%, 90%, 95%, 99%, or 100%.

### Compositions

Compositions useful in the methods of the present disclosure include compositions that specifically recognize an antigen on brain-derived exosomes, wherein the antigen is CD9, CD63, CD81 or CD171, EAAT1, or OMG. In yet other embodiments, the composition is selected from the group consisting of a peptide, a nucleic acid, an antibody, and a small molecule.

In certain embodiments, the present disclosure relates to compositions that specifically detect a brain-derived exosome. As detailed elsewhere herein, the disclosure is based upon the finding that CD9 and CD171 are specific antigens for subpopulations of brain-derived exosomes that may be used in the diagnosis or prognosis of brain damage from surgery. In some embodiments, the compositions of the present disclosure specifically bind to and detect CD9, CD171, SNAP25, EAAT1, OMGP, DR1, SR2A, SR2C, GABAB1, KOR, OR, and DAT. In other embodiments, the compositions of the present disclosure specifically bind to and detect a receptor for dopamine, serotonin, GABA, glutamate, opioid, orexin, adrenalin, noradrenalin, acetylcholine, and/or dopamine transporter.

In some embodiments, the composition comprises an antibody, where the antibody specifically binds to an antigen found on brain-derived exosomes. The term “antibody” as used herein and further discussed below is intended to include fragments thereof which are also specifically reactive with a brain-derived exosome. Antibodies can be fragmented using conventional techniques and the fragments screened for utility in the same manner as described above for whole antibodies. For example, F(ab) 2 fragments can be generated by treating antibody with pepsin. The resulting F(ab)₂ fragment can be treated to reduce disulfide bridges to produce Fab fragments. Antigen-binding portions may also be produced by recombinant DNA techniques or by enzymatic or chemical cleavage of intact antibodies. Antigen-binding portions include, inter alia, Fab, Fab′, F(ab′)2, Fv, dAb, and complementarity determining region (CDR) fragments, single-chain antibodies (scFv), single domain antibodies, bispecific antibodies, chimeric antibodies, humanized antibodies, diabodies and polypeptides that contain at least a portion of an immunoglobulin that is sufficient to confer specific antigen binding to the polypeptide. In certain embodiments, the antibody further comprises a label attached thereto and able to be detected (e.g., the label can be a radioisotope, fluorescent compound, enzyme or enzyme co-factor).

In certain embodiments, an antibody of the disclosure is a monoclonal antibody, and in certain embodiments, the disclosure makes available methods for generating novel antibodies that specifically bind the brain-derived exosome of the disclosure. For example, a method for generating a monoclonal antibody that specifically binds a brain-derived exosome, may comprise administering to a mouse an amount of an immunogenic composition comprising the brain-derived exosome, or fragment thereof, effective to stimulate a detectable immune response, obtaining antibody-producing cells (e.g., cells from the spleen) from the mouse and fusing the antibody-producing cells with myeloma cells to obtain antibody-producing hybridomas, and testing the antibody-producing hybridomas to identify a hybridoma that produces a monoclonal antibody that binds specifically to the brain-derived exosome. Once obtained, a hybridoma can be propagated in a cell culture, optionally in culture conditions where the hybridoma-derived cells produce the monoclonal antibody that binds specifically to the brain-derived exosome. The monoclonal antibody may be purified from the cell culture.

The term “specifically reactive with” as used in reference to an antibody is intended to mean, as is generally understood in the art, that the antibody is sufficiently selective between the antigen of interest (e.g., a brain-derived exosome) and other antigens that are not of interest. In certain methods employing the antibody, such as therapeutic applications, a higher degree of specificity in binding may be desirable. Monoclonal antibodies generally have a greater tendency (as compared to polyclonal antibodies) to discriminate effectively between the desired antigens and cross-reacting polypeptides. One characteristic that influences the specificity of an antibody:antigen interaction is the affinity of the antibody for the antigen. Although the desired specificity may be reached with a range of different affinities, generally preferred antibodies will have an affinity (a dissociation constant) of about 10⁻⁶, 10⁻⁷, 10⁻⁸, 10⁻⁹ or less.

Antibodies can be generated to bind specifically to an epitope of an exosome of the disclosure, including, for example, neuron-derived exosomes, astrocyte-derived exosomes, and oligodendrocyte-derived exosomes. In some embodiments the neuron-derived exosomes are pre-synaptic dopaminergic neuron-derived exosomes, or post-synaptic dopaminergic, serotonergic, GABAnergic, glutamatergic, and opioid neuron derived exosomes.

In addition, the techniques used to screen antibodies in order to identify a desirable antibody may influence the properties of the antibody obtained. A variety of different techniques are available for testing interaction between antibodies and antigens to identify particularly desirable antibodies. Such techniques include ELISAs, surface plasmon resonance binding assays (e.g., the Biacore binding assay, Biacore AB, Uppsala, Sweden), sandwich assays (e.g., the paramagnetic bead system of IGEN International, Inc., Gaithersburg, Md.), western blots, immunoprecipitation assays, immunocytochemistry, and immunohistochemistry.

In some embodiments, the present disclosure relates to compositions used for preventing and/or treating brain damage in subjects undergoing surgery. As detailed elsewhere herein, the present disclosure is based upon the findings that CD171, CD9, CD61, CD81, SNAP25, EAAT1, OMG, DR1, SR2A, SR2C, GABAB1, GluR-1, KOR, OR, and DAT are cell surface markers for specific subpopulations of brain-derived exosomes.

Therefore, in one embodiment, the present disclosure provides compositions that are useful for detecting and/or quantifying subpopulations of brain-derived exosomes during or following surgery.

### Methods of Treatment

The compositions and methods of the disclosure are also useful for preventing, minimizing, or treating damage to a brain during surgery. the disclosure provides a method for preventing, minimizing, or treating damage to a brain during surgery, comprising: (i) obtaining a biological sample from a patient that is undergoing or has undergone surgery, (ii) detecting whether a brain-derived exosome is present in the sample by contacting the sample with an antibody and detecting binding between the brain-derived exosome and the antibody, and (iii) administering an effective amount of a compound to prevent, minimize, or treat damage to the brain. In some embodiments, the biological sample is selected from the group consisting of whole blood, serum, plasma, urine, interstitial fluid, peritoneal fluid, cervical swab, tears, saliva, buccal swab, skin, brain tissue, and cerebrospinal fluid. In other embodiments, the compound is selected from the group consisting of an anti-inflammatory compound, an anti-oxidative stress compound, a free radical scavenger (e.g., edaravone), and a neuronal growth and survival factor compound. In yet other embodiments, the compound is administered before, during, or after the surgery. In still other embodiments, the compound is administered if brain-derived exosomes are detected in the sample. In some embodiments, the compound is administered if brain-derived exosomes are increased or reduced in the sample compared to a control. In other embodiments, the exosomes are selected from the group consisting of neuron-derived exosomes, astrocyte-derived exosomes, oligodendrocyte-derived exosomes, and microglia-derived exosomes. In still other embodiments, the biological sample is selected from the group consisting of whole blood, serum, plasma, urine, interstitial fluid, peritoneal fluid, cervical swab, tears, saliva, buccal swab, skin, brain tissue, and cerebrospinal fluid.

In some embodiments, a neuroprotective agent is used in the methods of the disclosure. Neuroprotective agents that may be used in the methods of the present disclosure are described in Panahi et al. (2018) Journal of Pharmacopuncture; 21(4):226-240. In some embodiments, the neuroprotective compound is a glutamate blocker (e.g., polyaginine R18, NA-1), a statin (e.g., atorvastatin), a hormone (e.g., melatonin), a hematopoietic growth factor (e.g., erythropoietin), a free radical scavenger (e.g., tempol), an immunosuppressant (e.g., cyclosporin A), a mucolytic agent (e.g., NAC), a beta-adrenergic receptor blocker (e.g., carvedilol), a COX-2 inhibitor (e.g., celecoxib), and/or an herbal medicine (e.g., curcumin).

### Kits

Another aspect of the disclosure encompasses kits for detecting or measuring brain damage in a subject undergoing or having undergone surgery. A variety of kits having different components are contemplated by the current disclosure. Generally speaking, the kit will include the means for quantifying one or more brain-derived exosomes in a biological sample obtained from the subject. In another embodiment, the kit will include means for collecting a biological sample, means for quantifying one or more brain-derived exosomes in the biological sample, and instructions for use of the kit contents. In certain embodiments, the kit comprises a means for enriching or isolating brain-derived exosomes in a biological sample. In further aspects, the means for enriching or isolating exosomes comprises reagents necessary to enrich or isolate brain-derived exosomes from a biological sample. In certain aspects, the kit comprises a means for quantifying the amount of a brain-derived exosome or a specific type of brain-derived exosome (e.g., neural-derived exosome). In further aspects, the means for quantifying the amount of brain-derived exosomes comprises reagents necessary to detect the amount of the brain-derived exosomes.

These and other embodiments of the present disclosure will readily occur to those of ordinary skill in the art in view of the disclosure herein.

### Examples

The disclosure will be further understood by reference to the following examples, which are intended to be purely exemplary of the disclosure. These examples are provided solely to illustrate the claimed disclosure. The present disclosure is not limited in scope by the exemplified embodiments, which are intended as illustrations of single aspects of the disclosure only. Any methods that are functionally equivalent are within the scope of the disclosure. Various modifications of the disclosure in addition to those described herein will become apparent to those skilled in the art from the foregoing description. Such modifications are intended to fall within the scope of the appended claims.

### Example 1: Isolation and Quantification of Brain-Derived Exosomes from Biological Samples from Subjects that have Undergone Cardiac Surgery

Brain-derived exosomes (BDE) were isolated and quantified from biological samples from subjects that had undergone cardiac surgery as follows. Forty seven (47) subjects with transcatheter aortic valve implantation (TAVI) and 18 subjects with total arterial revascularization (TAR) were recruited in this study after institutional review board (IRB) was approved at National Cerebral and Cardiovascular Center (NCCC) in Japan. EDTA blood was taken pre- and post-operation in the same day, followed by Day 1, 2, and 5, and plasma was isolated and frozen in −80° C. freezer. These samples were shipped to a laboratory in a dry ice package for neuron derived exosome (NDE) analysis.

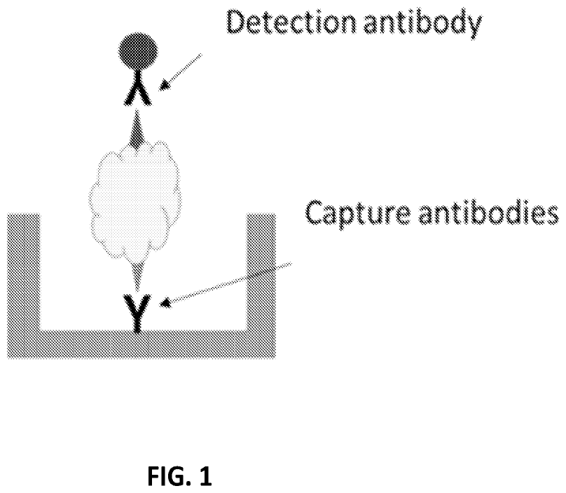

An exemplary assay principle is shown in FIG. 1. Plasma samples (5 uL diluted in a buffer in a final volume of 40 uL) were applied to enzyme-linked immunosorbent assay (ELISA) wells, where antibody against neurons (CD171=L1CAM) were previously immobilized. After CD171+ biomolecules were captured, unbound materials were removed by extensive washing steps, followed by a reaction with a biotinylated anti-CD9 antibody. Next, CD171+CD9+ double positive biomolecules (neuron-derived exosomes, NDEs) were detected by chemiluminescent ELISA with horseradish peroxidase-conjugated streptavidin and biotin-tyramide amplifications. Because exosomes are sticky and bind to IgG nonspecifically, plasma samples were also applied to ELISA wells where control mouse IgG was immobilized. For the CD9 reaction, excess amounts of control mouse IgG were included to eliminate such non-specific reaction. Mouse IgG was used as a control, because both anti-CD171 and CD9 were mouse monoclonal antibodies.

NDE in pre-, post, and Day 1-5 samples were analyzed in a single ELISA plate, and post-surgery changes (% Change) were calculated by dividing each ELISA reading (relative light units (RLU)) with RLU of pre-operation values. In order to consolidate the data from multiple ELISA plates a comparable dilution of a standard plasma control sample was assigned as 100 units/ml and was used in each ELISA plate and RLU was converted to U/mL.

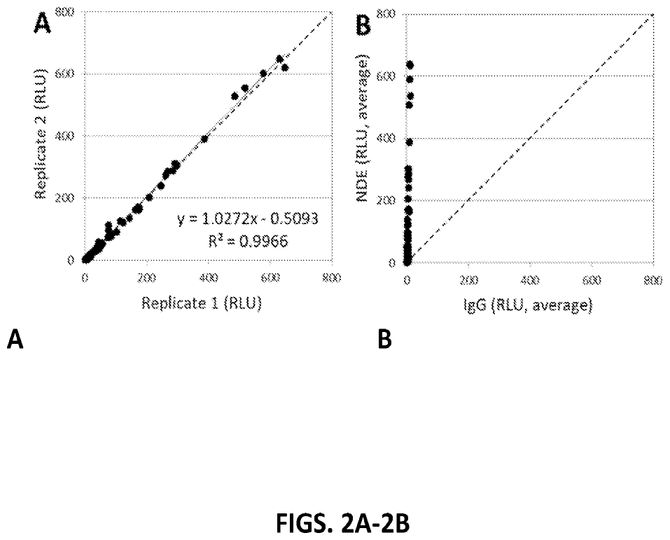

As shown in FIG. 2A, duplicate variation of CD171⁺CD9⁺ double positive values (NDE) were small with r²=0.9966. The non-specific binding to IgG (FIG. 2B, x-axis) was very small compared with NDE values (FIG. 2B, y-axis). Therefore, in subsequent studies, NDE values were analyzed without IgG.

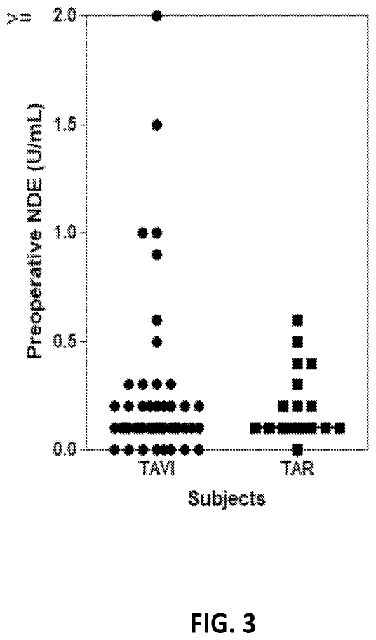

As shown in FIG. 3, plasma levels of NDE in pre-operative samples were not different between TAVI and TAR groups.

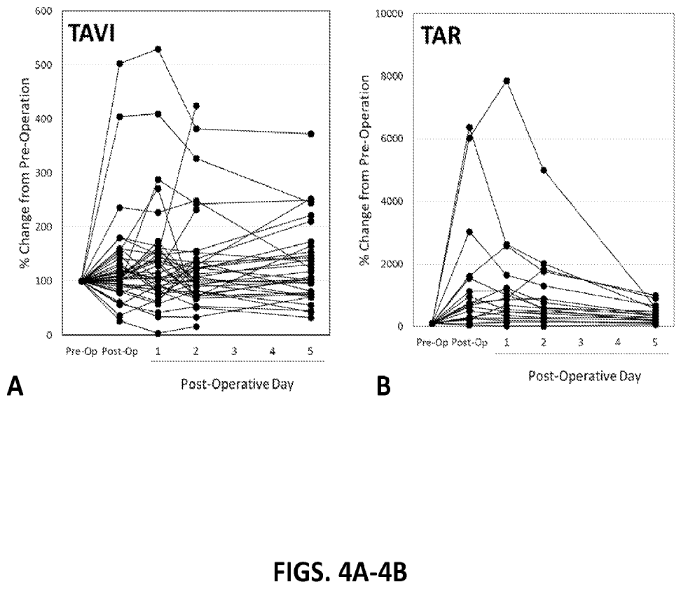

As shown in FIG. 4A, post-operative NDE values for 41 TAVI subjects were stable and less than 200%, whereas 6 subjects showed >200% increase (12.5%). In contrast, 16 TAR subjects showed >200% (88.9%) and only 2 subjects failed to show >200% increase (FIG. 4B). The increase was substantially higher in TAR than TAVI and 4 TAR subjects showed >1000% (10 folds) increase (FIG. 4B). Interestingly, such increases in both TAVI and TAR were transient and gradually decreased. However, even after Day 5, 5 out of 38 TAVI (13.2%) and 13 out of 17 TAR (76.5%) subjects still showed >200% increases.

These results showed that plasma levels of NDE are significantly increased in subjects after TAVI and TAR surgery. The results demonstrated that the methods and compositions of the disclosure may be used to quantitate and detect brain-derived exosomes in samples from subjects following surgery. These results further suggested that the methods and compositions of the disclosure would be useful for measuring brain damage associated with surgery. These results also suggested that compositions and methods of the disclosure would be useful for preventing and/or treating brain damage in subjects undergoing surgery.

### Example 2: Prediction of Post-Operative Delirium by Measuring Brain-Derived Exosomes in Peripheral Blood

Brain-derived exosomes (BDE) were isolated and quantified from biological samples from subjects that had undergone cardiac surgery to predict post-operative delirium as follows. Plasma samples were collected before operation from subjects for less invasive transcatheter aortic valve replacement (TAVI) (n=30) and invasive open-heart surgery, total aortic valve replacement (TAR) (n=29), respectively. Each patient was followed up for at least 5 days to observe delirium. In the first set of study, pre-, post-, 1, 2, and 5 days after operation (5 points per subjects) from 11 TAR subjects who did not show POD and 7 TAR subjects who developed POD were analyzed. In a subsequent study, pre-operative plasma samples from all subjects (30 TAVI and 29 TAR) were analyzed.

EDTA-plasma samples used for standard and assay controls were purchased from three different commercial sources (Innovative Research, Novi, MI, BioIVT, Westbury, NY, and Equitech Enterprise, Kerrville, TX). Antibodies against human CD171 (Thermo Fisher Scientific, Waltham, MA), EAAT1 (Excitatory Amino Acid Transporter 1) (Abcam, Waltham, MA), MOG (Myelin Oligodendrocyte Glycoprotein) (Thermo Fisher), CD9 (biotinylated, BD Biosciences, Franklin Lakes, NJ), and control mouse IgG (Equitech), 96 well plate for enzyme-linked immunosorbent assay (ELISA) (Sigma Aldrich, St Louis MO), ELISA coating buffer (BioLegend, San Diego, CA), and various reagents for ELISA (Thermo Fisher Scientific) were also purchased from designated suppliers.

Anti-CD171, anti-EAAT1, and anti-MOG were immobilized to ELISA wells. Plasma samples were diluted in plasma diluents in a final volume of 40 ul, then applied to the ELISA wells, and incubated for 1 hour at room temperature. After extensive washing steps with PBS, each well was reacted with biotinylated probes supplemented with 0.8% bovine serum albumin, 40 ug/mL mouse IgG, and reaction was continued for another 1 hour. After washing each well with PBS twice, each well was reacted with 1/4,000 dilution of poly-horseradish peroxidase (HRP)-conjugated streptavidin supplemented with 10% BSA and 30% blocker casein, and incubation was continued for 20 min. After washing steps, each well was incubated for 5 min with 0.0006% hydrogen peroxide (CVS pharmacy, Irvine, CA) diluted in PBS: water (1:1) solution to remove non-specifically bound HRP conjugates. After aspiration of hydrogen peroxidase, each well was mixed with 1/3 dilution of chemiluminescent substrate (Super Signal) for 4 min, then relative light units (RLU) were determined by a luminometer (Active GLO, ANSH Labs, Webster, TX). Using our standard plasma, arbitrarily assigned to 100 units/mL (U/mL), ELISA readings of RLU were converted to U/mL by 4 parameter logistic formula.

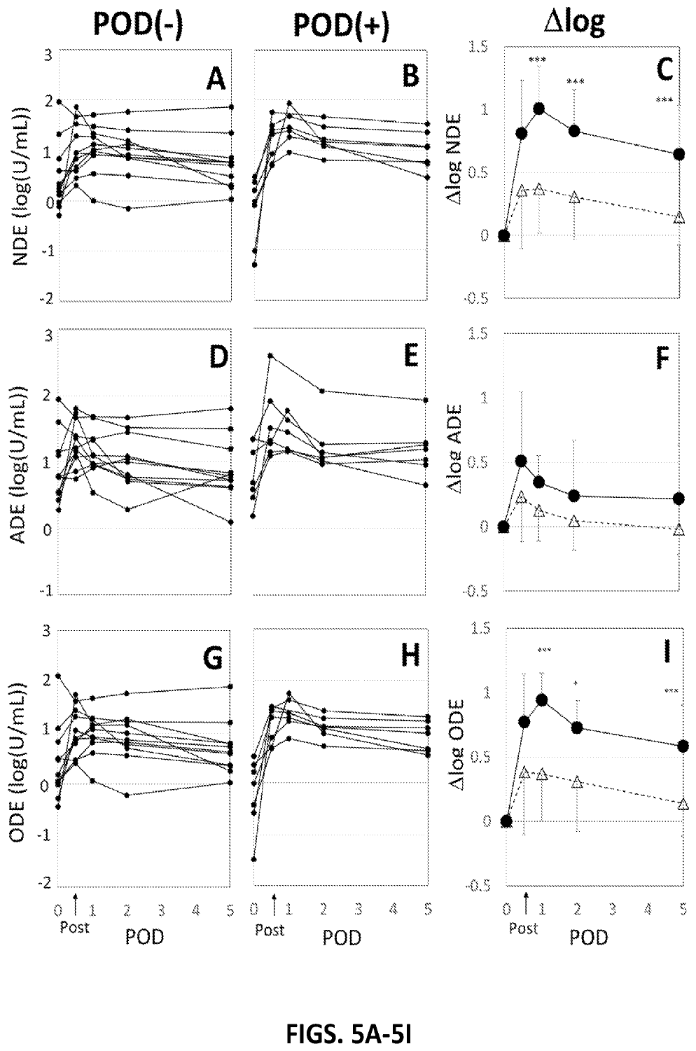

Plasma levels of NDE were widely spread among subjects, and such spread was also seen in ODE (FIGS. 5A, 5B, 5G, and 5H, log scale in y-axis). When the changes (Δ log) of NDE, ADE, and ODE were calculated (FIGS. 5C, 5F, and 5I), plasma levels of NDE and ODE were largely increased after TAR operation, but not ADE, and such increases of NDE and ODE were significantly higher in POD subjects than in non-POD subjects. When data from each patient were analyzed (FIGS. 5A, 5B, 5D, 5E, 5G, and 5H), the post-operative levels of NDE and ODE were quite similar between POD (−) and POD (+) groups, whereas pre-operative values were slightly lower in POD (+) compared to POD (−) patient groups, although no statistical significance was found due to the limited number of subjects.

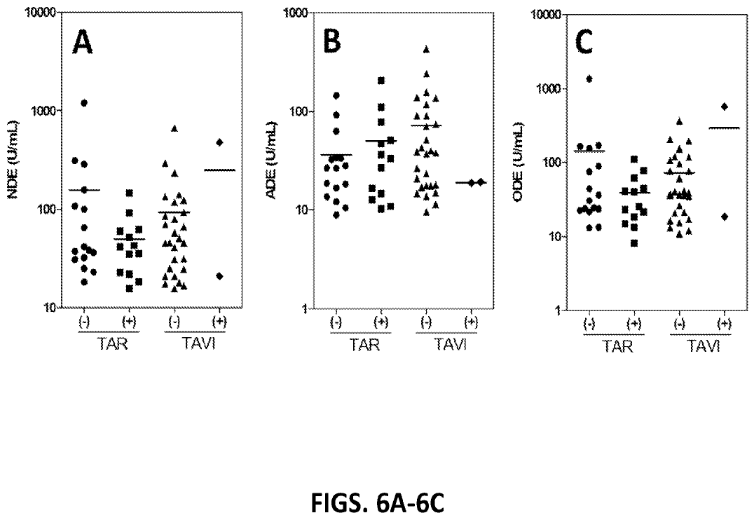

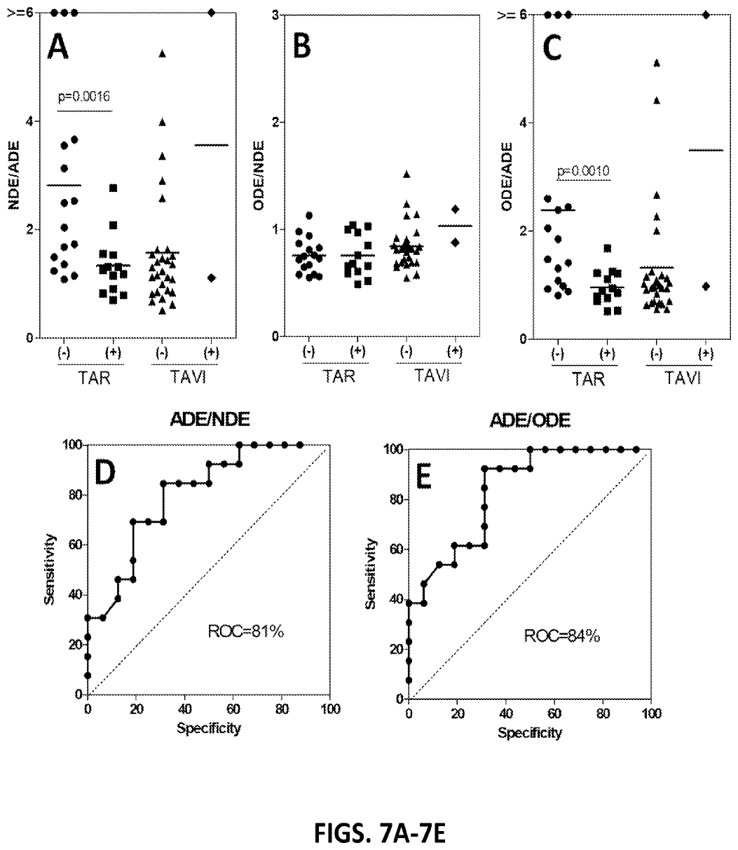

In subsequent studies, we focused on the preoperative values of NDE, ADE, and ODE, with an increased number of TAR subjects (n=29), and including additional TAVI subjects (n=30). Approximately half (n=13) of TAR subjects developed POD, whereas only two subjects of TAVI showed POD. As shown in FIGS. 6A-6C, pre-operative values of NDE (FIG. 6A) and ODE (FIG. 6C) were lower in POD(+) subjects than POD(−). However, after calculating the ratio of NDE/ADE, ODE/NDE and ODE/ADE, the spread was eliminated (FIGS. 7A-7C, linear scale in Y-axis). When POD(−) and (+) subjects were combined, TAVI and TAR groups showed similar NDE/ADE, ODE/NDE, and ODE/ADE, respectively (FIGS. 7A-7C). However, in the TAR group, NDE/ADE (FIG. 7A) and ODE/ADE (FIG. 7C) were significantly lower (p=0.0016 for NDE/ADE and p=0.001 for ODE/ADE) in POD(+) subjects than POD(−) subjects. These results showed that subjects with lower levels of NDE/ADE and ODE/ADE prior to surgery are more likely to develop POD with area under curve (AUC)>81% by receiver operator characteristic (ROC) analysis (FIGS. 7D-7E). Such low NDE/ADE and ODE/ADE subjects did not induce POD when less invasive TAVR was employed.

These results showed that plasma levels of NDE and ODE are significantly increased in subjects after TAR surgery. NDE and ODE in POD(+) subjects were significantly higher than POD(−) subjects (FIGS. 5A-5I). The results demonstrated that the methods and compositions of the disclosure may be used to quantitate and detect brain-derived exosomes in samples from subjects following surgery. These results also showed that the methods and compositions of the disclosure are useful for predicting post-operative delirium. These results further suggested that the methods and compositions of the disclosure would be useful for measuring brain damage associated with surgery. These results also suggested that compositions and methods of the disclosure would be useful for preventing and/or treating brain damage in subjects undergoing surgery.

### Example 3: Monitoring of Post-Brain Injuries Using a Plasma Based Immunoassay Between Neuron Marker CD171 and Exosome Marker CD9

Monitoring of post-brain injuries using a plasma-based immunoassay between neuron marker CD171 and exosome marker CD9 was carried out as follows.

Mouse monoclonal antibody against human CD171 (IgG2a, Thermo Fisher Scientific, Waltham, MA) and CD81 (BD Biosciences, Franklin Lakes, NJ), control mouse IgG2a (BioLegend, San Diego, CA), and normal mouse IgG (Equitech-Bio, Kerrville, TX) were diluted in ELISA coating buffer (BioLegend), and 50 uL was applied to high capacity white 8-well strips (Corning #3923, Sigma Aldrich, St Louis MO). After 1 hour incubation with constant shaking at 700 rpm (shaking was always applied during incubation), each well was washed once with phosphate buffered saline (PBS, Thermo Fisher), and incubated with undiluted blocker casein (Thermo Fisher) for another 1 hour.

Plasma samples were diluted in plasma diluents in a final volume of 40 uL, then applied to ELISA wells, where appropriate antibodies and control IgG were previously immobilized. ELISA wells were incubated for 1 hour at room temperature. After extensive washing steps with PBS, each well was reacted with biotinylated probes supplemented with 0.8% bovine serum albumin (BSA, Thermo Fisher), 40 ug/mL mouse IgG (Equitech-Bio), and the reaction was continued for another 1 hour. In order to maintain captured exosomes intact, tween-20 was not used at any time during the procedure. Biotinylation was carried out by the vendor's protocol using EZ link Sulfo-NHS-LC-Biotin (Thermo Fisher), followed by the spin column procedure to remove free biotin. After washing each well with PBS twice, each well was reacted with 1/4,000 dilution of poly-horseradish peroxidase (HRP)-conjugated streptavidin (Thermo Fisher) supplemented with 10% BSA (Equitech-Bio) and 30% blocker casein (Thermo Fisher), and incubation was continued for 20 min. After washing steps, each well was incubated for 5 min with 0.0006% hydrogen peroxide (CVS pharmacy, Irvine, CA) diluted in PBS:water (1:1) solution to remove non-specifically bound HRP conjugates. After aspiration of hydrogen peroxidase, each well was mixed with 1/3 dilution of chemiluminescent substrate (Super Signal Thermo Fisher) for 4 min, then relative light units (RLU) were determined by a luminometer (Active GLO, ANSH Labs, Webster, TX). Using our standard plasma, arbitrarily assigned to 100 units/mL (U/mL), ELISA readings of RLU were converted to U/mL by 4 parameter logistic formula.

After exosomes were captured and washed, each ELISA bottom was moved by a puncher, and suspended in 2% glutaraldehyde in 0.1M sodium phosphate buffer (PBS), pH 7.4 (Sigma Aldrich). For conductive staining, well bottoms were treated with 1% tannic acid (Nacalai Tesque, Kyoto, Japan) in 0.1 M PBS for 1 hour, rinsed in the buffer for 1 hour and immersed in 1% osmium tetroxide (OsO4) in 0.1 M PBS for 1 hour. The specimens were then dehydrated through a graded ethanol series, transferred to t-butyl alcohol and dried in a freeze dryer (ID-2; Eiko Co., Tokyo, Japan). Subsequently, dried specimens were mounted onto aluminum bases and coated with platinum-palladium in an ion-sputter coater (E1030; Hitachi, Tokyo, Japan). Finally, the specimens were observed in a field emission scanning electron microscopy at an accelerating voltage of 5 kV (S 4100; Hitachi, Tokyo, Japan).

We first tested that [CD171⁺CD9⁺] was not irreversible but dissociated by washing with acid solution with pH 2.5-2.0. We also found appropriate paramagnetic beads (PM-50, Spherotech, Lake Forest, IL) with the same surface characteristic of ELISA wells, so that the procedure developed for ELISA can be immediately applied to paramagnetic beads. After the performance of the paramagnetic beads was validated, plasma samples were applied to the paramagnetic beads. Captured exosomes were then eluted by pH 2.5 solution and immediately neutralized by adding appropriate amounts of 1M Tris, pH 8.0. A high-resolution particle size distribution and concentration analyses were carried out at Particle Technology Labs (Downers Grove, IL) using NanoSight NS300.

Control EDTA-plasma samples were purchased from three different commercial sources (Innovative Research, Novi, MI, BioIVT, Westbury, NY, and Equitech Enterprise, Kerrville, TX). EDTA plasma was collected in accordance with Institutional Review Board approval and consent was obtained from each participant. Plasma samples from high school athletes and adult soccer players were collected from Indiana University, college rugby players were from Keio University (Japan). Plasma samples from subjects who underwent TAR were collected at five time points (pre-operation, post-operation, postoperative day 1, 2, and 5) at National Cerebral and Cardiovascular Center (Japan). All plasma samples were frozen and stored at −80° C. freezer until analysis.

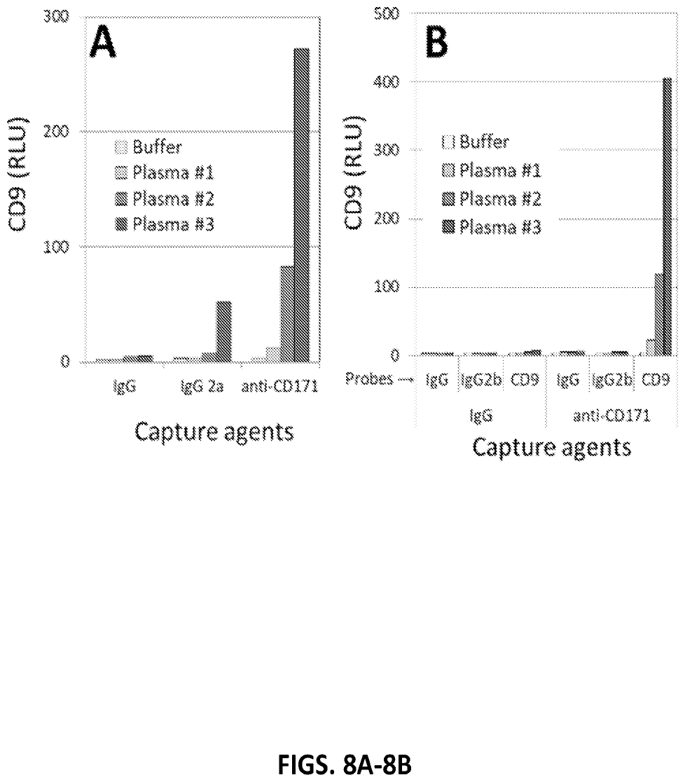

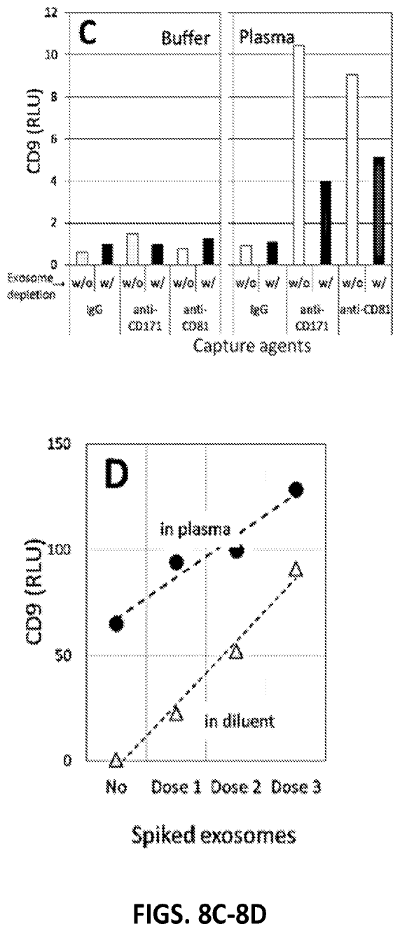

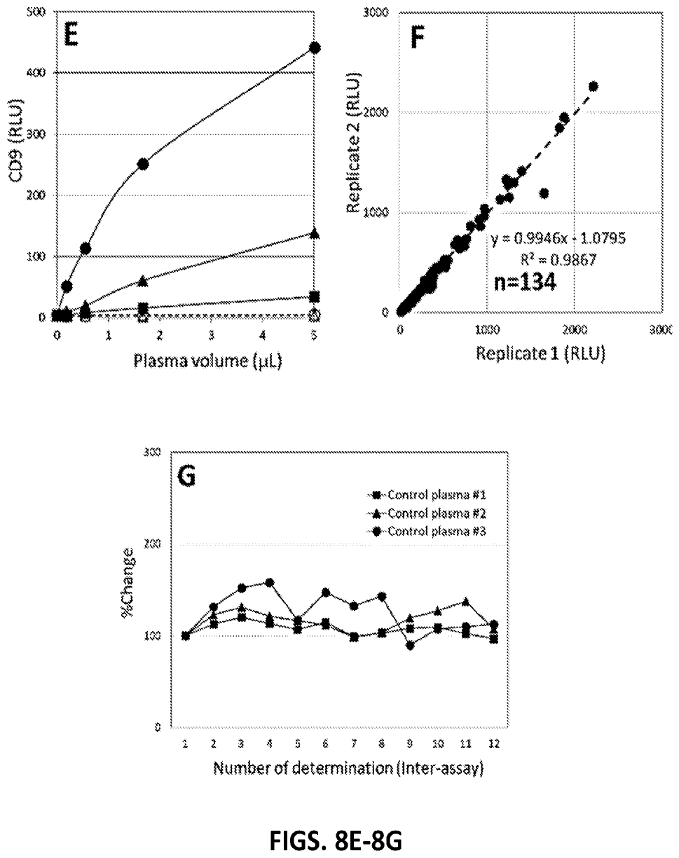

ELISA validation. Capture antibody specificity: Mouse monoclonal antibody (IgG2a) against human CD171, control mouse IgG, and control mouse IgG2a were immobilized on the ELISA wells. Three different control human plasma and diluents alone were applied to the designated wells, and ELISA was carried out using anti-human CD9 probes. As shown in FIG. 8A, all three plasma showed higher CD9 signals on anti-CD171-immobilized wells than control mouse IgG and isotype control mouse IgG wells. Although plasma #3 showed a limited signal in the IgG2a control wells, the levels on anti-CD171 were much higher than those of IgG2a. The #3 plasma is a good example that highlights the issue of the non-specific binding of IgG as well as the variation in plasma from different donors.

Probe specificity: Three control plasma and buffer alone were applied to mouse IgG or anti-CD171-immobilized ELISA wells, then reacted with biotinylated probes of mouse IgG, mouse IgG2b, or anti-human CD9 (mouse monoclonal IgG2b). As shown in FIG. 8B, all three plasma samples showed high anti-CD9 signals on anti-CD171-immobilized wells, but minimal signals from control wells, control probes, and buffer controls, demonstrating CD9 specificity.

Exosome specificity I: Exosome reduction. Plasma samples were incubated with anti-CD81− or control mouse IgG-immobilized paramagnetic beads, respectively, for 1 hour to remove exosomes. Beads were removed by magnetic separation and supernatant plasma samples were applied to 3 different ELISA wells, where anti-CD171, anti-CD81−, and control mouse IgG were previously immobilized. After extensive washing, wells were reacted with biotinylated anti-CD9 to complete the sandwich ELISA. As shown in FIG. 8C, [CD81+CD9+] exosomes in anti-CD81-treated plasma (w/, dark gray columns) were decreased by approximately 50% compared to the control IgG-treated plasma (w/o, light gray columns), indicating that approximately half of exosomes were removed by this procedure. [CD171+CD9+] exosomes were reduced by a similar degree by the treatment of exosome reduction, indicating that [CD171+CD9+] were exosomes.

Exosome specificity II: Spiked exosomes. Exosomes were prepared from control plasma using the ExoQuick (System Biosciences, Palo Alto, CA) procedure and suspended in PBS. Three different diluted exosomes and PBS were spiked into plasma or plasma diluents, and [CD171+CD9+] was determined. As shown in FIG. 8D (□), prepared exosomes showed [CD171+CD9+] in a dose-dependent manner. The addition of exosomes into plasma samples also increased [CD171+CD9+] in a dose-dependent manner with a similar slope to that of exosomes alone (●). These data indicate that [CD171+CD9+] is exosomes-dependent.

Plasma dilution studies: As shown in FIG. 8E, [CD171+CD9+] in 3 different plasma showed linear increases in a plasma volume-dependent manner on anti-CD171-immobilized wells (black-filled; ●▴▪), whereas signals stayed very low when samples were applied to control mouse IgG-immobilized ELISA wells (open symbols; ∘□□). These data support not only the specificity of the assay, but also that the results of ELISA data in each sample can be converted to U/mL using the dilution curve of standard plasma.

Assay reproducibility: The assay was always performed in duplicate, and as shown in FIG. 8F, intra-assay duplicate variation (n=134 subjects) was extremely small and inter-assay (n=8 runs) variation (FIG. 8G) remained between 90-158% variance from the 1st determination.

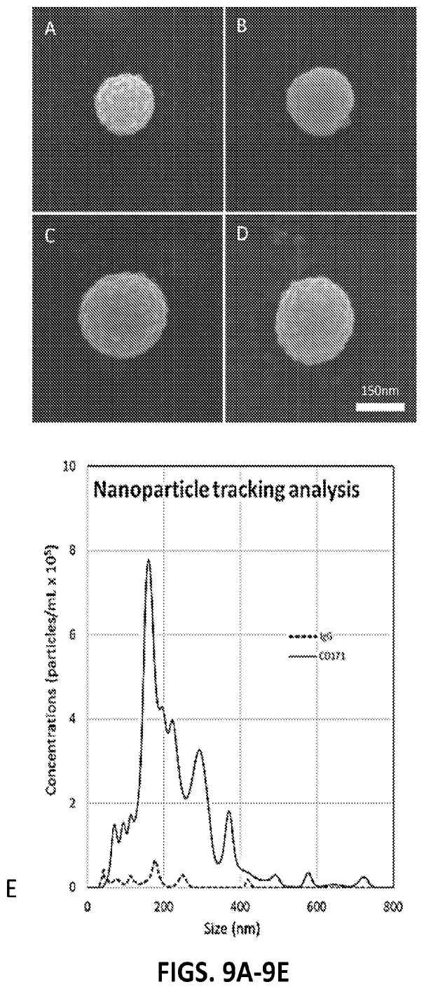

Confirmation of exosomes. SEM: Since SEM requires a flat surface, we removed each ELISA well bottom carefully without damaging the surface. We tested 2 different exosomes, NDE using anti-CD171 (FIGS. 9A-9B) and pan-exosomes using anti-CD81 (FIGS. 9C-9D) as capture agents, respectively. SEM images showed the visualization of exosomes with clear surface characteristics. All were sphere shape with a similar size of 100-200 nm. NDEs were slightly smaller than pan exosomes. Although FIGS. 9A-9D showed a single typical exosome in each panel, many similar figures were also seen.

NTA: Plasma samples were applied to anti-CD171− and control mouse IgG-immobilized paramagnetic beads. Captured exosomes were eluted by low pH 2.5 solution, neutralized, then applied to NTA as described in the Methods. As shown in FIG. 9E, the eluted solution from anti-CD171 immobilized paramagnetic beads showed 150-300 nm particles, equivalent to the size of SEM, whereas the solution from control IgG-immobilized beads failed to show such particles. Anti-CD171 also showed small peaks around 380, 500, 580, and 720 nm, indicating the presence of large molecules/aggregates in the sample.

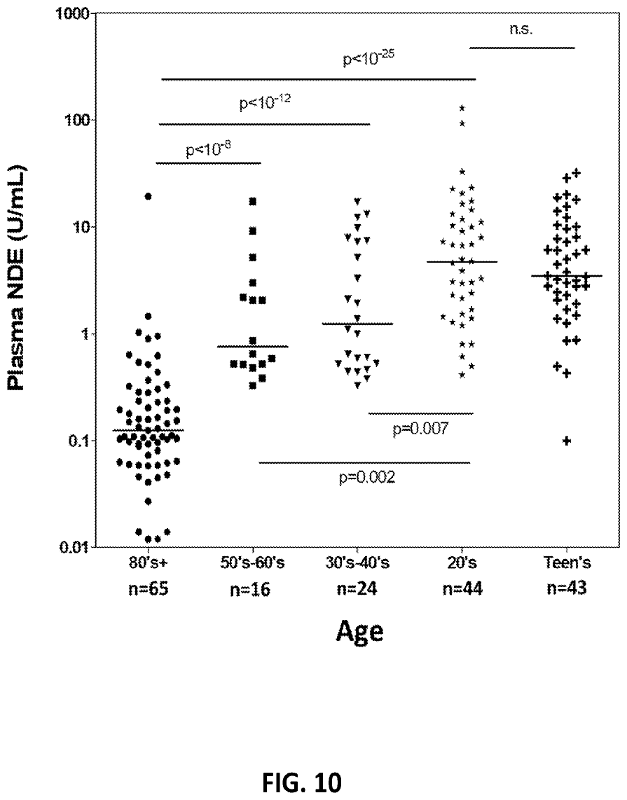

Range of control values. We collected plasma samples from various sources, ranging in age from teen's to >80's (total 192 subjects). These samples were collected from 3 different commercial sources and from different academic collaborators in both the US and Japan. All gender and ethnicity information were included. Surprisingly, [CD171⁺CD9⁺] was widely spread among individuals and such spread was almost 2-3 logs wide (FIG. 10). However, the variation was not random and showed normal-like distribution in log scale in each age group. Interestingly, the levels were significantly decreased by aging (FIG. 10).

Monitoring of the levels of [CD171⁺CD9⁺].

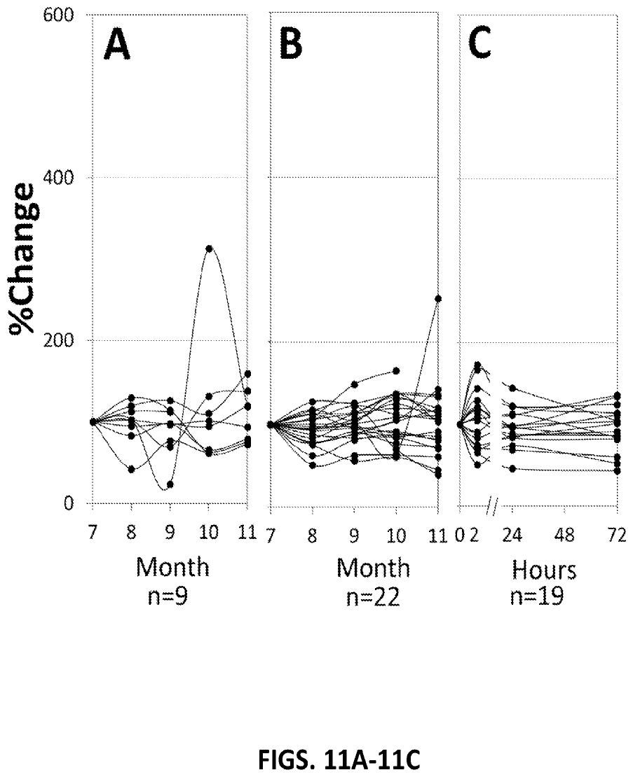

Controls: Although [CD171⁺CD9⁺] was widely different among individuals (FIG. 10), the values were stable within the same individual in high school cross-country athletes (non-contact sports, n=9) from July to November, and stayed within +/−50% from the values obtained in July, except one individual who showed a large fluctuation (FIG. 11A). This was similar to the results as reported in our previous publication (7), even though the assay had been improved substantially in the interim.

Contact sports athletes: High school American football athletes (n=22) were also stable from July to November (FIG. 11B). None of high school players suffered a concussion during this season. Adult soccer players (n=19) were also very stable for 3 days after intensive heading practice, although a small increase was seen 2 hours after the practice (FIG. 11C).

These results showed that plasma levels of brain-derived exosomes are significantly increased in subjects after TAR surgery. Brain-derived exosomes in POD(+) subjects was significantly higher than POD(−) subjects. The results demonstrated that the methods and compositions of the disclosure may be used to quantitate and detect brain-derived exosomes in samples from subjects following surgery. These results also showed that the methods and compositions of the disclosure are useful for predicting post-operative delirium. These results further suggested that the methods and compositions of the disclosure would be useful for measuring brain damage associated with surgery. These results also suggested that compositions and methods of the disclosure would be useful for preventing and/or treating brain damage in subjects undergoing surgery.

Various modifications of the disclosure, in addition to those shown and described herein, will become apparent to those skilled in the art from the foregoing description. Such modifications are intended to fall within the scope of the appended claims.

All references cited herein are hereby incorporated by reference herein in their entirety.

What is claimed is:

1. A method comprising: (i) obtaining a biological sample from a subject that is undergoing or has undergone surgery, and (ii) detecting brain-derived exosomes in the sample by contacting the sample with antibodies and detecting binding between the brain-derived exosomes and the antibodies.

2. The method of claim 1, wherein the detecting binding between the brain-derived exosomes and the antibodies further comprises contacting the brain-derived exosomes with a second antibody.

3. The method of claim 1, wherein the antibodies are anti-CD171 antibodies, anti-EAAT1 antibodies, or anti-MOG antibodies.

4. The method of claim 2, wherein the second antibody is an anti-CD9 antibody, an anti-CD63 antibody, or an anti-CD81 antibody.

5. The method of claim 1, further comprising quantifying the levels of brain-derived exosomes in the biological sample.

6. The method of claim 1, wherein the exosomes are selected from the group consisting of neuron-derived exosomes, astrocyte-derived exosomes, oligodendrocyte-derived exosomes, and microglia-derived exosomes.

7. The method of claim 1, further comprising determining a ratio of two exosomes selected from the group consisting of neuron-derived exosomes, astrocyte-derived exosomes, oligodendrocyte-derived exosomes, and microglia-derived exosomes.

8. The method of claim 7, wherein a lower ratio of NDE/ADE and/or ODE/ADE prior to surgery indicates that the subject will have postoperative delirium.

9. The method of claim 1, wherein the subject has been diagnosed or is suspected of having brain damage.

10. The method of claim 9, wherein the brain damage is postoperative cognitive dysfunction or decline (POCD).

11. The method of claim 9, wherein the brain damage is postoperative delirium (POD).

12. The method of claim 1, wherein the biological sample is selected from the group consisting of whole blood, serum, plasma, urine, interstitial fluid, peritoneal fluid, cervical swab, tears, saliva, buccal swab, skin, brain tissue, and cerebrospinal fluid.

13. The method of claim 1, wherein the surgery is cardiac surgery.

14. A method for measuring and/or detecting brain damage in a subject undergoing a surgical procedure comprising: (i) obtaining one or more biological samples from a subject before, during, and/or after undergoing surgery or any combination thereof, (ii) detecting brain-derived exosomes in the one or more samples by contacting the one or more samples with antibodies and detecting binding between the brain-derived exosomes and the antibodies, and (iii) comparing the levels of brain-derived exosomes in the one or more samples to a control.

15. The method of claim 14, wherein the levels of brain-derived exosomes are increased or reduced compared to a control.

16. A method for preventing, minimizing, or treating damage to a brain during surgery, comprising: (i) obtaining a biological sample from a subject that is undergoing or has undergone surgery, (ii) detecting whether a brain-derived exosome is present in the sample by contacting the sample with an antibody and detecting binding between the brain-derived exosome and the antibody, and (iii) administering an effective amount of a compound to prevent, minimize, or treat damage to the brain.

17. The method of claim 16, wherein the biological sample is selected from the group consisting of whole blood, serum, plasma, urine, interstitial fluid, peritoneal fluid, cervical swab, tears, saliva, buccal swab, skin, brain tissue, and cerebrospinal fluid.

18. The method of claim 17, wherein the compound is selected from the group consisting of an anti-inflammatory compound, an anti-oxidative stress compound, a free radical scavenger, and a neuronal growth and survival factor compound.

19. The method of claim 18, wherein the free radical scavenger is edaravone.

20. The method of claim 16, wherein the compound is administered before, during, or after the surgery.

21. The method of claim 16, wherein the compound is administered if brain-derived exosomes are detected in the sample.

22. The method of claim 16, wherein the compound is administered if brain-derived exosomes are increased or reduced in the sample compared to a control.

23. The method of claim 16, wherein the exosomes are selected from the group consisting of neuron-derived exosomes, astrocyte-derived exosomes, oligodendrocyte-derived exosomes, and microglia-derived exosomes.

24. The method of claim 16, wherein the biological sample is selected from the group consisting of whole blood, serum, plasma, urine, interstitial fluid, peritoneal fluid, cervical swab, tears, saliva, buccal swab, skin, brain tissue, and cerebrospinal fluid.

25. The method of claim 16, further comprising determining the ratio of at least two brain-derived exosomes in the biological sample.

26. The method of claim 25, wherein a lower ratio of NDE/ADE and/or ODE/ADE prior to surgery indicates that the subject will have or likely to have postoperative delirium

27. A kit for measuring brain damage in a subject undergoing or having undergone surgery, the kit comprising one or more agents which specifically bind brain-derived exosomes, one or more containers for collecting and or holding the biological sample, and an instruction for its use.

28. The kit of claim 27, wherein the agent is an anti-CD9 antibody, an anti-CD63 antibody, an anti-CD81 antibody, an anti-EAAT1 antibody, an anti-OMG antibody, and/or an anti-CD171 antibody.

29. The kit of claim 27, wherein the exosomes are selected from the group consisting of neuron-derived exosomes, astrocyte-derived exosomes, oligodendrocyte-derived exosomes, and microglia-derived exosomes.

30. The kit of claim 27, wherein the surgery is cardiac surgery.
